(19) United States
(12) Patent Application Publication
(10) Pub. No.: US 2009/0177287 A1
(43) Pub. Date: Jul. 9, 2009

(54) Endoprosthesis for Orthopedic Applications

(75) Inventors: Giuseppe Sala, Desio (Milano) (IT); Paolo Guerra, Milano (IT)

Correspondence Address: SHOEMAKER AND MATTARE, LTD, 10 POST OFFICE ROAD - SUITE 100, SILVER SPRING, MD, 20910, US

(73) Assignee: SINTEA BIOTECH S.P.A., Baranzate, Milano (IT)

(21) Appl. No.: 12/295,326

(22) PCT Filed: Mar. 31, 2006
(86) PCT No.: PCT/IT06/00206

§ 371 Date: Jan. 13, 2009

(51) Int. Cl. A61F 2/36

(52) U.S. Cl. 623/23.12

(57) ABSTRACT

An endoprosthesis for orthopaedic applications comprising a prosthesis body suitable for interfacing between a first and a second bone at a first and a second wall respectively. The prosthesis body, at least one of said walls, comprises a fixing interface suitable for favouring the osteo-integration with an associable bone, which is spaced from one of the walls of the prosthesis body by a plurality of spacer elements for forming a meatus between the interface and the wall, said meatus determining cavities and undercuts suitable for seating growing bony tissue. The fixing interface comprises a reticular structure having a plurality of meshes integrally interconnected to each other and suitable for favouring the osteo-integration.

### FIELD OF THE INVENTION

The present invention relates to endoprostheses for orthopaedic applications; in particular, the present invention relates to a prosthesis for cotyloid cavity, suitable for seating the head of a femur for ensuring the functionality of the coxofemoral articulation.

Endoprostheses for orthopaedic applications have the function of replacing or cooperating with the components of the skeletal system in their main function, of standing mechanical stresses. In both cases (of replacement or “collaboration”), endoprostheses must as soon as better as possible “integrate” with the newly formed bony tissue, that is, cause the so-called arthrodesis, that is, the fusion between endoprosthesis and bone. In the art, such object is for example achieved by the use of osteo-conductive materials, which allow the taking root and proliferation of the bony tissue. The best solutions are represented by titanium, or by titanium alloys with aluminium-vanadium or niobium-zirconium.

The use of osteo-inductive coatings that stimulate the growth and proliferation of the bony tissue is also known in the art. The best solutions are represented by bioglasses and hydroxy-apatite.

The use of surfaces having morphologies suitable for the incorporation and/or mechanical grip of the bony tissue is also known. The best solutions adopted so far consist in making rough surfaces, obtained by “plasma pore” techniques or by sintering metal micro-balls.

The above solutions of the prior art are not alternative, but they can be put into effect at the same time, thus making endoprostheses of osteo-conductive material, having rough surface coated with osteo-inductive coating.

If on the one side the selection of the osteo-conductive material and of the osteo-inductive coating has determined “legitimate” solutions, the excellent method for obtaining a sufficiently rough surface for favouring the bony tissue gripping has not been found yet.

In fact, all the techniques currently used imply the coating of the prosthesis body with a layer added at a later time through plasma or sintering techniques. This causes a discontinuity and thus weakness of the interface between substrate and coating. Moreover, such coatings are not capable of forming effective holds for the growing bone, and therefore bring to an effective steady and irreversible osteo-integration.

These problems characterise all endoprostheses for orthopaedic applications and in particular, those endoprostheses that in the normal use are subject to considerable stresses.

The coxofemoral articulation is very complex and is one of the most stressed articulations in the human body.

The cotyloid cavity prostheses of the prior art, comprising a spherical cap suitable for receiving the head of a femur and being fixed by screws to the seating of a cotyloid cavity, do not ensure a quick, steady and lasting gripping at the cotyloid cavity.

A quick and complete osteo-integration is very important since the femur head, as seen, is stressed by considerable forces.

The quickness of the osteo-integration allows the patient to recover the use of the limb very quickly, avoiding or reducing long and difficult periods of rehabilitation consequent to prolonged stasis and immobility.

### SUMMARY OF THE INVENTION

The problem of the present invention is to provide a prosthesis which should solve the disadvantages mentioned with reference to the prior art.

Such disadvantages are solved with a prosthesis in accordance with claim 1.

Other embodiments of the prosthesis according to the invention are described in the subsequent claims.

### BRIEF DESCRIPTION OF THE DRAWINGS

Further features and the advantages of the present invention will appear more clearly from the following description of preferred non-limiting embodiments thereof, wherein:

FIG. 1 shows a perspective view of a prosthesis for cotyloid cavity according to an embodiment of the present invention;

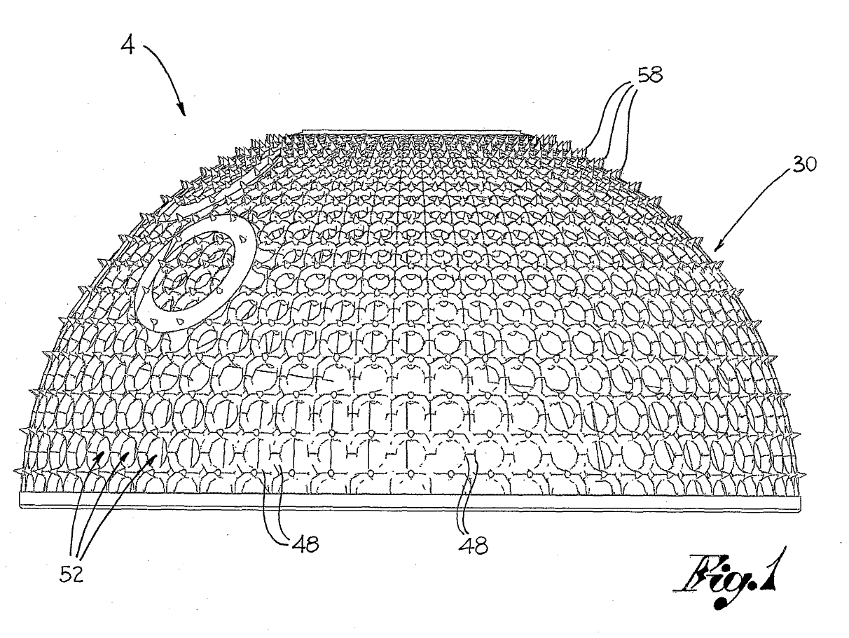

FIG. 2 shows a section view of the prosthesis of FIG. 1, along the section line II-II of FIG. 1;

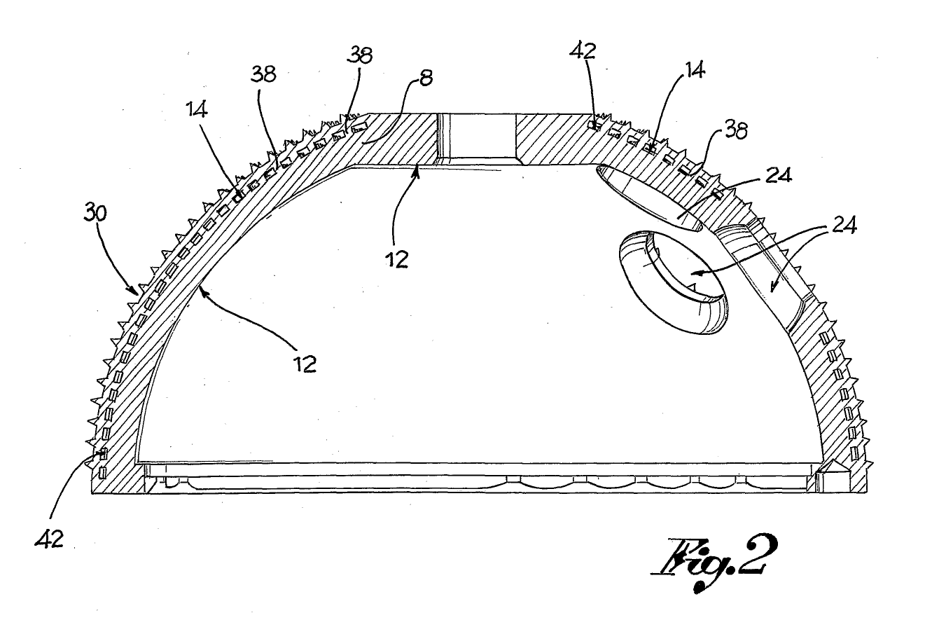

FIG. 3 shows a perspective view of the prosthesis of FIG. 1 in a configuration where it is mounted on a femur head.

### DETAILED DESCRIPTION OF THE INVENTION

Elements or parts of elements in common between the embodiments described below are referred to with the same reference numerals.

With reference to the above figures, reference numeral 4 generically denotes an orthopaedic endoprosthesis.

According to an embodiment, prosthesis 4 comprises a prosthesis body 8 suitable for interfacing between a first and a second bone or portions of bone. The prosthesis body 8 has the function of providing the necessary mechanical stiffness to the connection between the first and the second bone, that is, of absorbing the forces exchanged between the bones.

The prosthesis body 8 is provided with a first wall 12 suitable for interfacing with the first bone and a second wall 14 suitable for interfacing with the second bone.

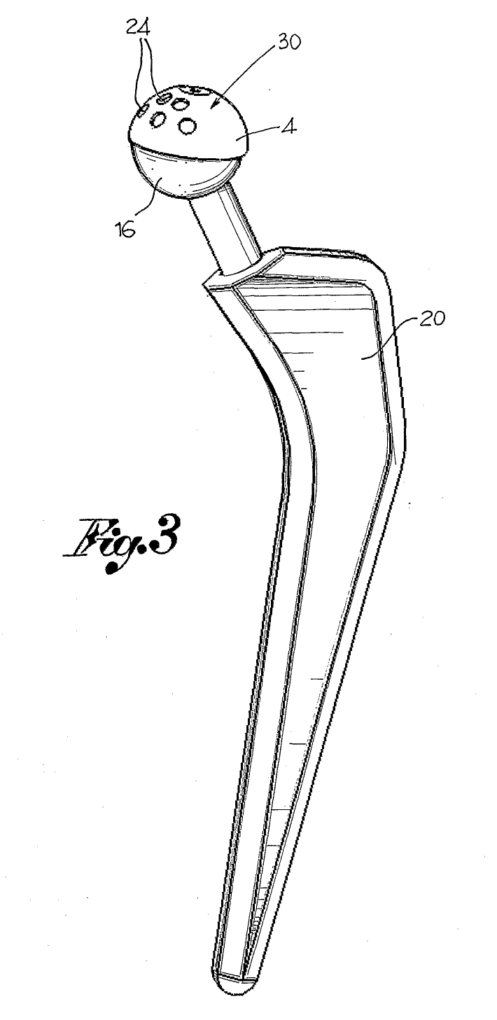

According to an advantageous embodiment (FIG. 3), prosthesis 4 is a prosthesis for cotyloid cavity, wherein the first bone is head 16 of a femur 20, whereas the second bone is a cotyloid cavity.

Prosthesis 4 comprises means for fixing the prosthesis body 8 suitable for obtaining a fixing of prosthesis 4 to at least one between the first and the second bone.

According to an embodiment, said fixing means comprise at least one connection hole 24 passing through the prosthesis body and at least one threaded connection means, such as a screw, suitable for passing through the connection holes 24 and for being screwed to the bone, for example in the cotyloid cavity.

The prosthesis body 8, at least one of said walls 12, 14, comprises a fixing interface 30 suitable for favouring the osteo-integration with an associable bone; in other words, the fixing interface 30 has the function of favouring the bony growth so as to ensure the osteo-integration of the prosthesis.

The fixing interface 30 is spaced from one of walls 12, 14 of the prosthesis body 8 by a plurality of spacer elements 38 integrally connected to the prosthesis body 8 for forming a meatus 42 between interface 30 and the relevant wall 12, 14, said meatus 42 determining cavities and undercuts suitable for seating growing bony tissue.

The fixing interface is integrally associated to the prosthesis body; preferably, the fixing interface 30 is integral with the prosthesis body 8.

The fixing interface 30 comprises a reticular structure having a plurality of meshes 48 integrally interconnected to each other. Meshes 48 are stiffly connected to each other and thus, they cannot move or rotate relative to the prosthesis body 8.

Advantageously, meshes 48 have a circular pattern, that is, they are comparable to discs, preferably circular. Preferably, meshes 48 comprise a central hole 52 so as to take on a ring configuration.

According to further embodiments, the meshes can take on different configurations, for example elliptical, triangular or quadrangular, preferably provided with central hole 52.

Advantageously, meshes 48 are rings having a circular section, relative to a section plane perpendicular to the associable wall 12, 14 of the prosthesis body 8. In other words, the meshes can be seen as rings formed by a ‘wire’ having circular section. According to further embodiments, the mesh section may be quadrangular or elliptical.

Preferably, meshes 48 are evenly distributed on the fixing interface 30, according to a regular and repetitive arrangement.

Meshes 48 may all have the same dimensions and shape, so as to form a homogeneous and even reticular structure on the entire interface 30. According to further embodiments of the present invention, the reticular structure may comprise meshes 48 having different dimensions and shape.

Prosthesis 4, at each mesh 48 comprises at least one spacer element 38 integrally connected to the prosthesis body 8.

According to an embodiment, the spacer elements 38 are equal to each other and perpendicular to the relevant wall 12, 14 of the prosthesis body 8, so that meatus 42 between the prosthesis body and interface 30 is substantially constant. Moreover, interface 30 is counter-shaped relative to the profile of the underlying wall 12, 14 of the prosthesis body 8.

The spacer elements 38 are preferably perpendicular to walls 12, 14 of the prosthesis body 8 and have a column configuration. Preferably, the spacer elements have a circular section, but they may also have an elliptical, polygonal or quadrangular section. Moreover, the spacer elements may be cylindrical, that is, have a constant section along the extension thereof, or they may have a variable section, for example decreasing from wall 12, 14 towards meshes 48.

According to a further embodiment, the spacer elements 38 are different from each other, for example having different heights, so as to create variable meatus 42 between the prosthesis body 8 and the fixing interface 30.

Preferably, the fixing interface 30, on the side opposite to the prosthesis body 8, comprises a plurality of prickles 58 suitable for sticking at least partly into the associable bone for improving the primary stability of the prosthesis.

Said prickles 58 are for example arranged on the fixing interface 30 at the spacer elements 38 and opposite thereto relative to the interface. According to an embodiment, prickles 58 have a conical configuration for favouring the fixing to the bone. Preferably, prickles 58 have a height comprised between 0.1 mm and 1.5 mm, and even more preferably, said prickles 58 have a height comprised between 0.2 mm and 1.0 mm.

According to an advantageous embodiment of the present invention, prosthesis 4 is a prosthesis for cotyloid cavity having in all a spherical cap configuration, wherein the prosthesis body 8 is suitable for seating head 16 of a femur 20 at the first wall 12 according to a rotary-translatory coupling.

Prosthesis 4 for cotyloid cavity comprises, at the second wall 14, opposite the first wall 12, a fixing interface 30 shaped as a spherical cap, suitable for being associated to a cotyloid cavity.

The present invention is not limited to the use of prostheses for cotyloid cavity, but extends to all the possible types and shapes of endoprostheses whose purpose is to speed up and optimise the osteo-integration process, such as:

in the field of major prosthetics: femoral cotyloid cavities and stems for hip bone prostheses, tibial plates, tibial and femoral stems for knee prostheses, glenoid components and humerus stems for shoulder prostheses;

in the field of prosthetics for the vertebral column: intersomatic spacers, somatic cages, disk prostheses, vertebral plates, bars and screws;

in the field of traumatology: synthesis plates for long bones, for hand and skull micro-surgery.

The above examples are not exhaustive and represent some of the possible advantageous uses of the present invention.

The present invention relates to endoprostheses made of different metals and metal alloys, such as (but not only): aluminium, copper, hafnium, lead, nickel, niobium, rhenium, stainless steels, tantalum, tin, titanium, tungsten, zinc, chromium, cobalt, molybdenum+all the possible combinations of these metals. At present, the most extensive and advantageous applications consist of titanium and alloys thereof, stainless steels, chromium-cobalt-molybdenum alloys, and nickel-chromium-cobalt-molybdenum alloys.

The prostheses may also be made of ceramics (at present alumina—aluminium oxide—and zirconia—zirconium oxide, but not only).

Moreover, the endoprostheses may also be made of polymers (at present PEEK—poly-ether-ether-ketone—and UHMWPE—high molecular weight polyethylene, but not only).

Preferably, meshes 48 exhibit a circular section having a diameter comprised between 0.1 mm and 0.5 mm; and even more preferably, said meshes 48 exhibit a circular section having a diameter equal to 0.2 mm.

Preferably, meshes 48 exhibit a circular ring pattern having a diameter comprised between 0.5 mm and 2 mm; even more preferably, said meshes 48 exhibit a circular ring pattern having a diameter equal to 1.0 mm.

Preferably, meatus 42 is comprised between 0.05 mm and 1 mm; and even more preferably, said meatus 42 is comprised between 0.1 mm and 0.5 mm.

It has been found that the above dimensional values ensure the best results in terms of bio-mimesis and osteo-integration.

A possible method of manufacture of a prosthesis according to the present invention shall now be described.

Preferably, the technology for manufacturing the prostheses in accordance with the present invention belongs to the category of FFF (Free Form Fabrication) techniques, or additional production methods which do not use dies or moulds. In particular, the technique preferably used is named EBM (Electron Beam Melting). Conceptually, it follows the rapid prototyping techniques, from which it differs in that it produces “finished” components capable of performing the functional tasks they are intended for, absolutely irrespective of the level of morphological complication.

In brief, the shape of the item to be manufactured (as complex, difficult, connected or not connected as desired) described by a CAD file is “cut” into thin slices (the more complex the shape, the thinner the slices). Such slices are then divided on their plane into “blocks” (similar to the “finished elements”), which are smaller as the complexity of the shape increases.

The shape, divided into “slices” and “blocks” is the information provided to a numerical control machine (a sort of three-dimensional plotter), whose “tool” consists of an electron beam.

This electron beam is piloted in a vacuum chamber inside which subsequent layers (corresponding to the above “slices”) of very fine material powder are prepared. For each “slice” or layer, the electron beam moves according to a path dictated by the division into “blocks”.

The specific energy (that is, energy by surface unit) of the electron beam is so high as to cause the instant melting and the immediate re-consolidation of the material. Where the electron beam does not pass, the material remains in the form of powder and is then eliminated, so as to leave the spaces occupied thereby empty.

As an alternative, among the FFF techniques it is also possible to use the selective laser-sintering which, similarly to the EBM technology, consists in addressing a laser beam having a specific energy sufficient for causing the instant and local melting of the portion of powder material impinged thereby.

In particular, the EBM technique is specifically used for making prostheses of titanium alloy, while the selective laser-sintering technique is for example used for making prostheses of Ch-Co based alloys.

As it can be understood from the description, the prosthesis according to the present invention allows overcoming the disadvantages of the prostheses of the prior art.

In particular, the prosthesis according to the invention allows facilitating the osteo-integration process, thanks to the creation of a plurality of cavities, interspaces and undercuts suitable for favouring the bone gripping to the prosthesis.

The particular circular shape of the mesh elements further favours the bony growth.

Moreover, the shape of the mesh wires in turn having circular section further favours the bony growth and grip, since it imitates the structure of the bony trabeculas.

In particular, the structures with circular section of the prosthesis according to the invention are geometrically similar to the bony trabeculas and thus improve the bio-mimesis and the osteo-integration. In particular, it has been found that the use of meshes with circular section, relative to a plane perpendicular to the fixing interface, favours the bony growth.

The prostheses according to the present invention ensure reduced bony growth and prosthesis osteo-integration times, as this is covered and incorporated in the grown bone.

Thanks to the presence of undercuts, the prosthesis anchoring is firm and steady over time and ensures high resistance even in applications on especially stressed bones, as in the case of the coxofemoral articulation.

The prostheses according to the present invention are particularly resistant since they are of the monolithic type, that is, they comprise a solid and compact prosthesis body, preferably of metal, having the function of standing the mechanical stresses to which a mesh element is integrally associated with the function of creating resistant and lasting bonds with the bone, by undercuts that facilitate the incorporation of the prosthesis itself.

Advantageously, the presence of the prickles on the outer surface of the mesh element facilitates and improves the primary stability of the prosthesis.

A man skilled in the art can make several changes and adjustments to the prostheses described above in order to meet specific and incidental needs. For example, a man skilled in the art could use osteo-conductive coatings, for example based on bioglasses or hydroxyapatite, for further stimulating the growth and proliferation of the bony tissue.

These and other variations fall within the scope of protection as defined by the following claims.

1-37. (canceled)

38. Endoprosthesis for orthopaedic applications comprising
a prosthesis body suitable for interfacing between a first and a second bone or portions of bone,
the prosthesis body being provided with a first wall suitable for interfacing with said first bone and a second wall suitable for interfacing with said second bone,
means for fixing the prosthesis body suitable for obtaining a fixing of the prosthesis to at least one between the first and the second bone,
the prosthesis body, at least one of said walls comprising a fixing interface suitable for favoring the osteo-integration with an associable bone,
wherein
the fixing interface is spaced from one of the walls of the prosthesis body by a plurality of spacer elements integrally connected to the prosthesis body for forming a meatus between the interface and the wall, said meatus determining cavities and undercuts suitable for seating growing bony tissue,
the fixing interface comprises a reticular structure having a plurality of meshes integrally interconnected to each other, the meshes having circular section relative to a section plane perpendicular to the associable wall of the prosthesis body.

39. An endoprosthesis according to claim 38, wherein said fixing interface is integrally associated to the prosthesis body.

40. An endoprosthesis according to claim 38, wherein said fixing interface is integral with the prosthesis body.

41. An endoprosthesis according to claim 38, wherein the meshes have a circular pattern.

42. An endoprosthesis according to claim 38, wherein the meshes comprise a central hole so as to take on a ring configuration.

43. An endoprosthesis according to claim 38, wherein said meshes have an elliptical configuration and are provided with a central hole.

44. An endoprosthesis according to claim 38, wherein the meshes are rings having circular section relative to a section plane perpendicular to the associable wall of the prosthesis body.

45. An endoprosthesis according to claim 38, wherein the meshes are evenly distributed on the fixing interface according to a regular and repetitive arrangement.

46. An endoprosthesis according to claim 38, wherein said prosthesis comprises at least one spacer element integrally connected to the prosthesis body so as to ensure at least one meatus between the prosthesis body and the fixing interface.

47. An endoprosthesis according to claim 46, wherein said prosthesis comprises a spacer element at each mesh.

48. An endoprosthesis according to claim 46, wherein said spacer elements are equal to each other and perpendicular to the relevant wall of the prosthesis body, so that the meatus between the prosthesis body and the interface is substantially constant.

49. An endoprosthesis according to claim 48, wherein the interface is counter-shaped relative to the profile of the underlying wall of the prosthesis body.

50. An endoprosthesis according to claim 46, wherein said spacer elements are different, so as to determine variable meatus between the prosthesis body and the interface.

51. An endoprosthesis according to claim 38, wherein said fixing means comprise a plurality of connection holes passing through the prosthesis body and suitable for seating screw connection means for fixing the prosthesis to a bone.

52. An endoprosthesis according to claim 38, wherein the fixing interface, on the side opposite to the prosthesis body, comprises a plurality of prickles suitable for sticking at least partly into the associable bone for ensuring the primary stability of the prosthesis.

53. An endoprosthesis according to claim 52, wherein said prickles are arranged on the fixing interface at the spacer elements and opposite thereto relative to the fixing interface.

54. An endoprosthesis according to claim 52, wherein said prickles have a conical configuration for favouring the fixing to the bone.

55. An endoprosthesis according to claim 52, wherein said prickles have a height comprised between 0.1 mm and 1.5 mm.

56. An endoprosthesis according to claims 55, wherein said prickles have a height comprised between 0.2 mm and 1.0 mm.

57. An endoprosthesis according to claim 38, wherein the prosthesis is a prosthesis for cotyloid cavity having in all a spherical cap configuration, wherein the prosthesis body is suitable for seating the head of a femur at the first wall according to a rotary-translatory coupling and on the second wall exhibits a fixing interface.

58. An endoprosthesis according to claim 57, wherein the fixing interface has a spherical cap pattern and is suitable for being associated to a cotyloid cavity.

59. An endoprosthesis according to claim 38, made of metals and metal alloys belonging to a group comprising aluminium, copper, hafnium, lead, nickel, niobium, rhenium, stainless steels, tantalum, tin, titanium, tungsten, zinc, chromium, cobalt, molybdenum.

60. An endoprosthesis according to claim 38, wherein the prosthesis is made of ceramic materials, such as alumina, aluminium oxide, zirconia, zirconium oxide.

61. An endoprosthesis according to claim 38, wherein the endoprostheses are made of polymers, such as poly-ether-ether-ketone (PEEK) and/or high molecular weight polyethylene (UHMWPE).

62. An endoprosthesis according to claim 38, wherein an osteo-conductive coating is applied to said interface, on the side of the associable bone, for further stimulating the growth and the proliferation of the bony tissue.

63. An endoprosthesis according to claim 62, wherein said osteo-conductive coating comprises bioglasses and/or hydroxy-apatite.

64. An endoprosthesis according to claim 38, wherein said meshes have a circular section having a diameter comprised between 0.1 mm and 0.5 mm.

65. An endoprosthesis according to claim 64, wherein said meshes have a circular section having a diameter equal to 0.2 mm.

66. An endoprosthesis according to claim 38, wherein said meshes exhibit a circular ring pattern having a diameter comprised between 0.5 mm and 2 mm.

67. An endoprosthesis according to claim 64, wherein said meshes exhibit a circular ring pattern having a diameter equal to 1.0 mm.

68. An endoprosthesis according to claim 38, wherein said meatus is comprised between 0.05 mm and 1 mm.

69. An endoprosthesis according to claim 68, wherein said meatus is comprised between 0.1 mm and 0.5 mm.

70. A method of manufacture of an endoprosthesis according to claim 38, said method comprising FFF (Free Form Fabrication) techniques, or additional production methods which do not use dies or molds.

71. A method according to claim 70, wherein said FFF technique is an EBM (Electron Beam Melting) technique comprising the steps of:
arranging powder material on a support, into a vacuum chamber,
addressing, according to the desired geometry, an electronic beam on the powder, said beam having a specific energy sufficient for causing the instant and local melting of a portion of powder material impinged thereby, so as to obtain the desired geometry,
removing the powder not impinged by the beam.

72. A method according to claim 71, wherein said powder comprises a titanium alloy.

73. A method according to claim 70, wherein said FFF technique is a selective laser-sintering technique comprising the steps of:
arranging powder material on a support, into a vacuum chamber,
addressing, according to the desired geometry, a laser beam on the powder, said laser beam having a specific energy sufficient for causing the instant and local melting of a portion of powder material impinged thereby, so as to obtain the desired geometry,
removing the powder not impinged by the laser beam.

74. A method according to claim 73, wherein said powder comprises a Ch-Co based alloy.
